(19) United States
(12) Patent Application Publication
(10) Pub. No.: US 2009/0292131 A1
(43) Pub. Date: Nov. 26, 2009

(54) PROCESSES FOR PREPARATION OF TAXANES AND INTERMEDIATES THEREOF

(76) Inventors: Ladislav CVAK, Opava-Zlatniky (CZ); Martin VALIK, Opava 5 (CZ); Tomás HOLAS, Chlebicov (CZ); Jirí MALECEK, Brno (CZ)

Correspondence Address: KENYON & KENYON LLP, ONE BROADWAY, NEW YORK, NY, 10004, US

(21) Appl. No.: 12/387,793

(22) Filed: May 6, 2009

Related U.S. Application Data
(60) Provisional application No. 61/126,861, filed on May 7, 2008.
(60) Provisional application No. 61/131,838, filed on Jun. 11, 2008.
(60) Provisional application No. 61/132,208, filed on Jun. 16, 2008.

(51) Int. Cl. C07D 305/14

(52) U.S. Cl. 549/510

(57) ABSTRACT

A paclitaxel intermediate of formula 1:

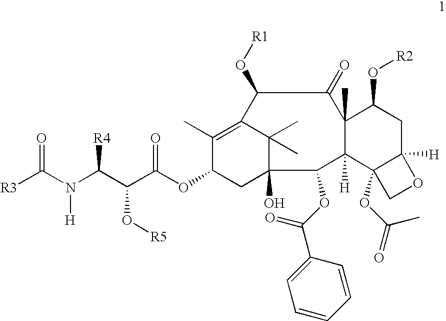

wherein R₁ is acetyl, R₂ is tert-butyloxycarbonyl (BOC), R₃ and R₄ are phenyl and R₅ is 1-ethoxyethyl, is provided.

Also provided are processes for preparing taxane intermediates of formula 1

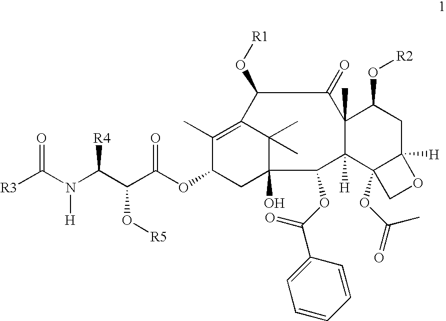

comprising reacting a compound of formula 2

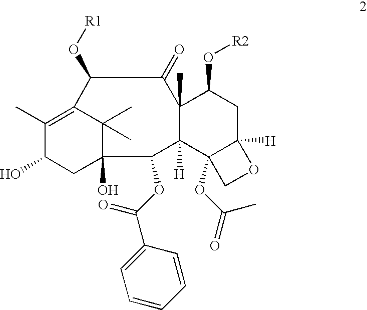

with a compound of formula 3

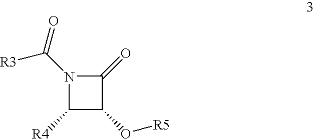

wherein,
R₁, R₂ and R₅ are independently a hydroxyl protecting group;
R₃ is phenyl, substituted phenyl, a straight or branched alkyl containing 1 to 12 carbon atoms, alkenyl containing 2 to 12 carbon atoms, cycloalkyl containing 4 to 15 carbon atoms, cycloalkenyl or an R₆—O— group in which R₆ is:
phenyl, substituted phenyl group, a C₁-C₈ straight or branched alkyl, a C₂-C₈ straight or branched alkenyl group, a C₃-C₈ straight or branched alkynyl, a C₃-C₇ cycloalkyl, C₄-C₇ cycloalkenyl, a C₇-C₁₁ bicycloalkyl substituent, or a saturated or unsaturated nitrogen; and
R₄ is phenyl or a substituted phenyl group.

### CROSS REFERENCE TO RELATED APPLICATIONS

This application claims the benefit of U.S. Provisional Patent Application Ser. No. 61/126,861, filed May 7, 2008; U.S. Provisional Patent Application Ser. No. 61/131,838, filed Jun. 11, 2008; and U.S. Provisional Patent Application Ser. No. 61/132,208, filed Jun. 16, 2008; the contents of which are incorporated by reference herein, in their entirety.

### FIELD OF THE INVENTION

The present invention relates to processes for the preparation of taxanes, especially preparation of paclitaxel from 7-Boc-2′-(1-ethoxyethyl)-paclitaxel, (“2′-EE-7-Boc paclitaxel”).

### BACKGROUND OF THE INVENTION

Paclitaxel (TAXOL®) of the following formula

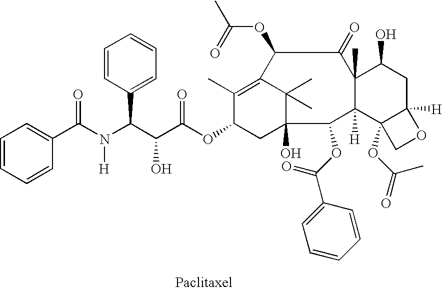

and docetaxel (TAXOTERE®) of the following formula

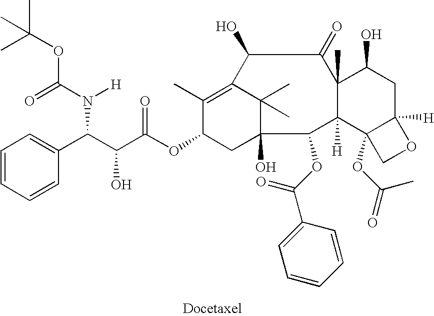

are used as cancer chemotherapeutic agents with a broad spectrum of antileukemic and tumor-inhibiting activities. The history and basic milestones of taxol and taxotere research, development and clinical testing are summarized in a book edited by M. Suffness (Taxol® Science and Applications, CRC Press, 1995).

Paclitaxel is a natural taxane, while docetaxel is a semisynthetic product. The primary source of paclitaxel was the bark of the pacific yew-tree Taxus brevifolia. Later on, paclitaxel was found in needles of many other types of yew (e.g., Taxus Canadensis and Taxus baccata, to name a few) creating thus a renewable source of this important drug. Besides paclitaxel, the needles of many types of yew contain significant amounts of baccatin III and/or 10-deacetyl-baccatin III (10-DAB) and these abundant natural taxanes became raw materials for partial synthesis of paclitaxel and docetaxel.

Docetaxel is prepared according to the process in U.S. Pat. No. 4,814,470 and demonstrated in the following scheme.

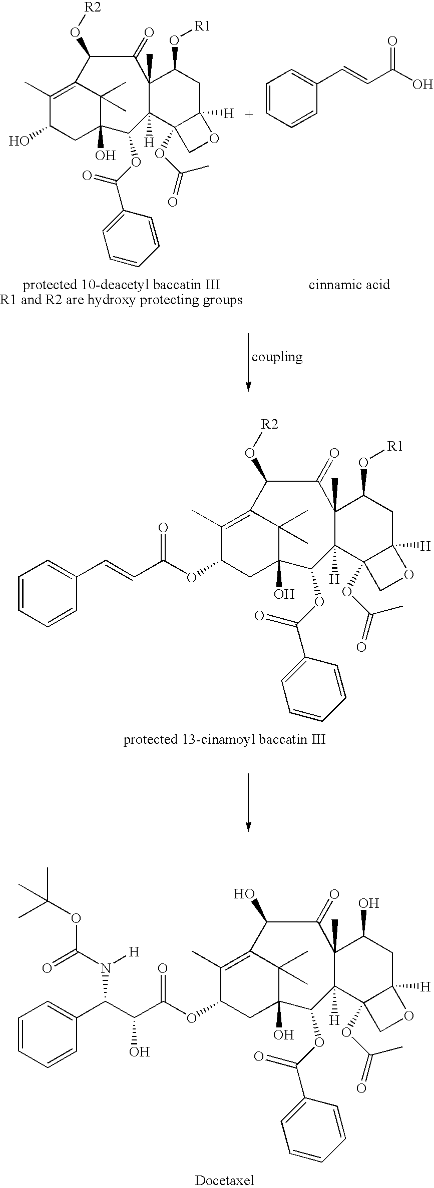

A semi-synthetic approach to paclitaxel and other taxanes is described in U.S. Pat. Nos. 4,924,011 and 4,924,012, and in the Journal of the American Chemical Society, 1988, 110, 5917. The synthesis is based on coupling of 7-protected derivatives of baccatin III with O-protected derivatives of N-benzoyl-phenylisoserine, using N,N′-dicyclohexylcarbodiimide (DCCI) or 2,2′-dipyridylcarbonate (DPC) as an activating agent; it is described in the following scheme from the Journal of the American Chemical Society, 1988, 110, 5917,

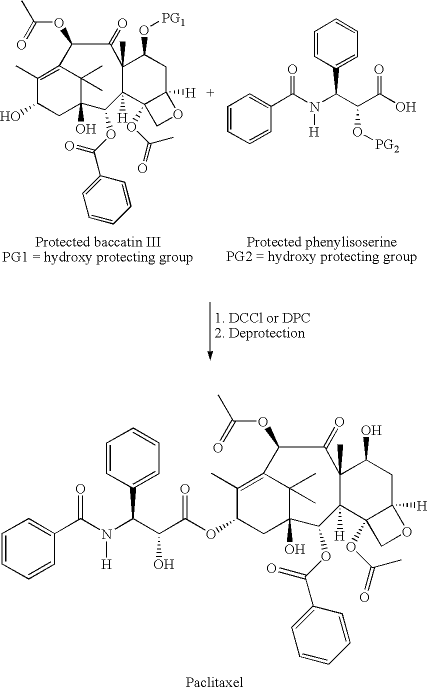

However, this route of synthesis requires a large excess of the protected phenylisoserine derivative.

U.S. Pat. No. 5,175,315 reports the coupling between 7-protected derivatives of baccatin III and a cyclic precursor of phenylisoserine, the β-lactam of formula I,

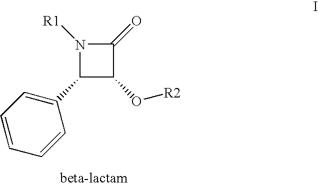

in pyridine, which is a toxic solvent, wherein R₁ is benzoyl or another acyl and R₂ is a hydroxyl protecting group.

U.S. Pat. Nos. 5,229,526, 5,274,124, and 5,430,160 report the coupling of the β-lactam of formula I using metal or quaternary ammonium alkoxide derivatives of 7-protected baccatin III and of 7,10-diprotected-10-deacetyl-baccatin III. The metal alkoxide derivatives of the baccatin compound are prepared by using an equimolar amount of an organometallic strong base at low temperatures of −30° C. to −78° C., in the presence of tetrahydrofuran (THF). Then, to the mixture containing the metal alkoxide of the baccatin compound is added the O-protected β-lactam of formula I, to obtain the 2′,7-diprotected taxane derivative, e.g., paclitaxel.

U.S. Pat. No. 6,187,916 reports a similar process but the equimolar amount of strong base is added to a mixture containing the O-protected β-lactam of formula I and 7-protected baccatin III or 7,10-diprotected-10-deacetyl-baccatin III.

In the above methods the obtained protected paclitaxel is purified by chromatography, which is a time-consuming operation. The invention described herein refers to improved processes for preparing taxanes, and in particular, for preparing paclitaxel via a new intermediate, 2′-EE-7-Boc paclitaxel, which are suitable for large-scale industrial production.

### SUMMARY OF THE INVENTION

In one embodiment, the present invention provides a paclitaxel intermediate, 7-Boc-2′-(1-ethoxyethyl)-paclitaxel, (2′-EE-7-Boc paclitaxel), of formula 1:

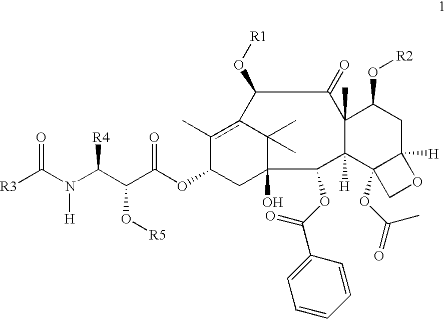

wherein R₁ is acetyl, R₂ is tert-butyloxycarbonyl (BOC), R₃ and R₄ are phenyl and R₅is 1-ethoxyethyl.

In another embodiment, the present invention provides the use of 2′-EE-7-Boc paclitaxel for the preparation of paclitaxel.

In yet another embodiment, the present invention provides a process for preparing 2′-EE-7-Boc paclitaxel comprising reacting 7-Boc-baccatin III of formula 2

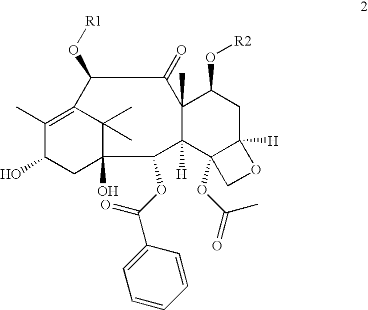

with a β-lactam of formula 3

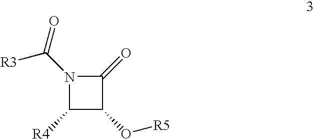

wherein R₁ is acetyl, R₂ is tert-butyloxycarbonyl (BOC), R₃ and R₄ are phenyl and R₅is 1-ethoxyethyl.

In one embodiment, the present invention provides a process for preparing paclitaxel comprising preparing 2′-EE-7-Boc paclitaxel according to the process of the present invention and converting it to paclitaxel.

In another embodiment, the present invention provides a process for preparing paclitaxel comprising reacting 2′-EE-7-Boc paclitaxel with formic acid or a mixture of formic acid and a second organic acid in the presence or absence of a solvent, wherein the solvent is immiscible in formic acid or in the mixture of formic acid and the second organic acid.

In another embodiment, the present invention provides a process for preparing a taxane intermediate of formula 1

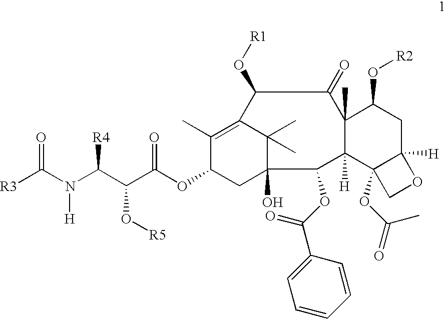

comprising reacting a 7-protected derivative of baccatin III or a 7,10-diprotected derivative of 10-deacetyl-baccatin III (10-DAB) of formula 2

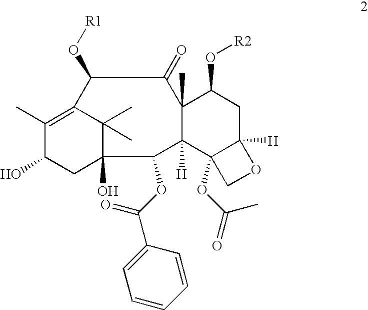

with a β-lactam of formula 3

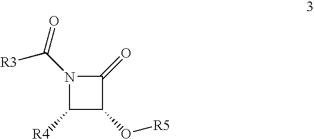

and a catalytic amount of a base chosen from a group consisting of an organometallic base, a metal hydride and mixtures thereof, providing a reaction mixture, and then quenching the reaction mixture, providing the intermediate of formula 1,
wherein,

R₁, R₂ and R₅ are independently a hydroxyl protecting group;

R₃ is phenyl, substituted phenyl, a straight or branched alkyl, alkenyl, cycloalkyl, cycloalkenyl or an R₆—O— group in which R₆ is:

phenyl, substituted phenyl group, a straight or branched alkyl, a straight or branched alkenyl group, a straight or branched alkynyl, a cycloalkyl, a cycloalkenyl, a bicycloalkyl substituent, or a saturated or unsaturated nitrogen; and

R₄ is phenyl or a substituted phenyl group.

In another embodiment, the present invention relates to preparing taxanes comprising preparing the taxane intermediate of formula 1 according to the process described herein and converting it into a taxane.

### BRIEF DESCRIPTION OF THE FIGURES

FIG. 1 shows a ¹H nuclear magnetic resonance (NMR) spectrum of 2′-EE-7-Boc paclitaxel.

FIG. 2 shows a ¹³C nuclear magnetic resonance (NMR) spectrum of 2′EE-7-Boc paclitaxel.

FIG. 3 shows a mass spectroscopy (MS) spectrum of 2′-EE-7-Boc paclitaxel.

FIG. 4 shows a powder X-ray diffraction (PXRD) spectrum of 2′-EE-7-Boc paclitaxel.

### DETAILED DESCRIPTION OF THE INVENTION

As used herein, “room temperature” refers to a temperature range of about 20° C. to about 30° C., preferably about 20° C. to about 25° C.

As used herein, “organic acid” refers to an organic compound with acidic properties, e.g., a carboxylic acid. In some embodiments, the organic acid may have a pKa of about 4.0 to about 6.0, preferably about 4.5 to about 5.0. Examples of organic acids include acetic acid and propionic acid.

As used herein, a solvent which is “immiscible” in formic acid or in a mixture of formic acid and another organic acid refers to a solvent that when combined with formic acid or with a mixture of formic acid and a second organic acid at a temperature of between about room temperature to about −15° C. forms a two-phase system.

As used herein, a “hydroxyl protecting group” refers to a group that is introduced in place of a hydroxyl group in a molecule by chemical modification of the hydroxyl group in order to obtain chemoselectivity in a subsequent chemical reaction, i.e., to prevent the hydroxyl group from participating in the subsequent chemical reaction. Examples of hydroxyl protecting groups include: tert-butyloxycarbonyl (BOC); acetyl (Ac); benzoyl (Bz); benzyl (Bn); β-methoxyethoxymethyl ether (MEM); dimethoxytrityl [bis-(4-methoxyphenyl)phenylmethyl, DMT]; methoxymethyl ether (MOM); methoxytrityl [(4-methoxyphenyl)diphenylmethyl, MMT); p-methoxybenzyl ether (PMB); methylthiomethyl ether; pivaloyl (Piv); tetrahydropyranyl (THP); trityl (triphenylmethyl) (Tr); silyl ether (e.g., trimethylsilyl (TMS), tert-butyldimethylsilyl (TBDMS), tert-butyldimethylsilyloxymethyl (TOM), and triisopropylsilyl (TIPS) ethers); methyl ethers; ethoxyethyl ethers (EE).

Unless indicated otherwise, “substituted phenyl” refers to phenyl substituted with chloro, bromo, fluoro, a C₁-C₁₂ straight or branched alkyl, a C₂-C₁₂ straight or branched alkenyl, a C₄-C₁₅ cycloalkyl, or a C₄-C₁₅ cycloalkenyl.

The present invention relates to processes for preparing taxanes, especially paclitaxel. The process for preparing paclitaxel is conducted via a new intermediate, 7-Boc-2′-(1-ethoxyethyl)-paclitaxel, (“2′-EE-7-Boc paclitaxel”), described in the following scheme 1:

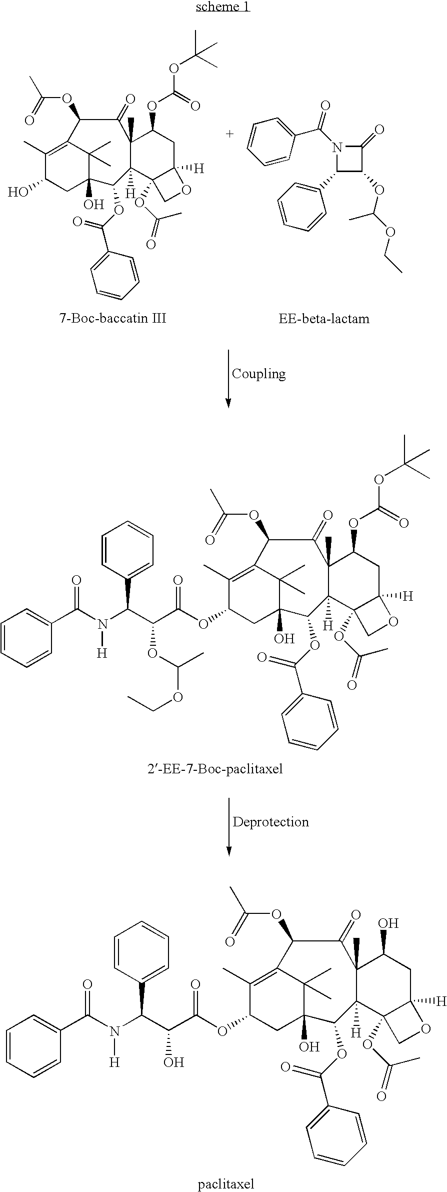

In 2′-EE-7-Boc paclitaxel, the two protecting groups, BOC and EE, are on one hand relatively stable and thus are not removed via side reactions producing impurities, and on the other hand are relatively labile under deprotection conditions, allowing clean formation of paclitaxel.

In this process, the coupling can be performed via two routes. In route A, solvents other than pyridine are used and thus, the current process is more environmentally friendly and safer for the operator. The use of solvents other than pyridine results also in a reaction mixture which is not viscous, in contrast with that of the prior art (see Comparative Example 6 herein), thus providing the product in high conversion even after a shorter reaction time. In addition, the use of solvents other than pyridine at this step results in a direct precipitation of the paclitaxel intermediate, thus simplifying the isolation of the intermediate.

The process according to route B applies a catalytic amount of a base selected from the group consisting of an organometallic base and a metal hydride, at room temperature, while the prior art processes use a stoichiometric amount of the base at very low temperatures, which are not convenient for industrial large-scale manufacturing. In addition, the recovery of the crude product in the process of the present invention is simple and thus more suitable for large-scale manufacturing. In addition, paclitaxel is obtained in higher yields than those reported previously

In one embodiment, the present invention provides a paclitaxel intermediate, 7-Boc-2′-(1-ethoxyethyl)-paclitaxel, (2′-EE-7-Boc paclitaxel), of formula 1:

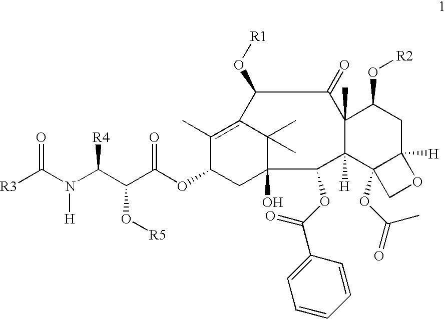

wherein R₁ is acetyl, R₂ is tert-butyloxycarbonyl (BOC), R₃ and R₄ are phenyl and R₅is 1-ethoxyethyl.

Preferably, the above 2′-EE-7-Boc paclitaxel is provided in an isolated form. Preferably, the isolated 2′-EE-7-Boc paclitaxel is solid. More preferably, it is crystalline.

As used herein, the term “isolated” in reference to 2′-EE-7-Boc paclitaxel corresponds to 2′-EE-7-Boc paclitaxel that is physically separated from the reaction mixture where it is formed. For example, the separation can be done by filtering the precipitated 2′-EE-7-Boc paclitaxel. More preferably the 2′-EE-7-Boc paclitaxel is separated from 7-Boc-baccatin III of formula 2

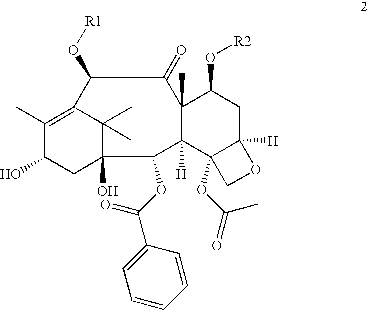

wherein R₁ is acetyl and R₂ is BOC,

providing a composition comprising 2′-EE-7-Boc paclitaxel and about 0% to about 5% of 7-Boc-baccatin III based on the combined weight of 2′-EE-7-Boc paclitaxel and 7-Boc-baccatin III in the composition, preferably, about 0% to about 2%, more preferably 0% to about 1% of 7-Boc-baccatin III, most preferably about 0% to about 0.5%, about 0% to about 0.2%, about 0% to about 0.1% of 7-Boc-baccatin III based on the combined weight of 2′-EE-7-Boc paclitaxel and 7-Boc-baccatin III in the composition.

Preferably, the provided composition comprises about 95% to about 100% of 2′-EE-7-Boc paclitaxel, more preferably about 98% to about 100%, most preferably, about 99.5% to about 100% 2′-EE-7-Boc paclitaxel by weight.

In other embodiments, the composition consists essentially of about 95% to about 100%, about 98% to about 100%, about 99.5% to about 100% of 2′-EE-7-Boc paclitaxel and about 5% to about 0% of 7-Boc-baccatin III based on the combined weight of 2′-EE-7-Boc paclitaxel and 7-Boc-baccatin III in the composition.

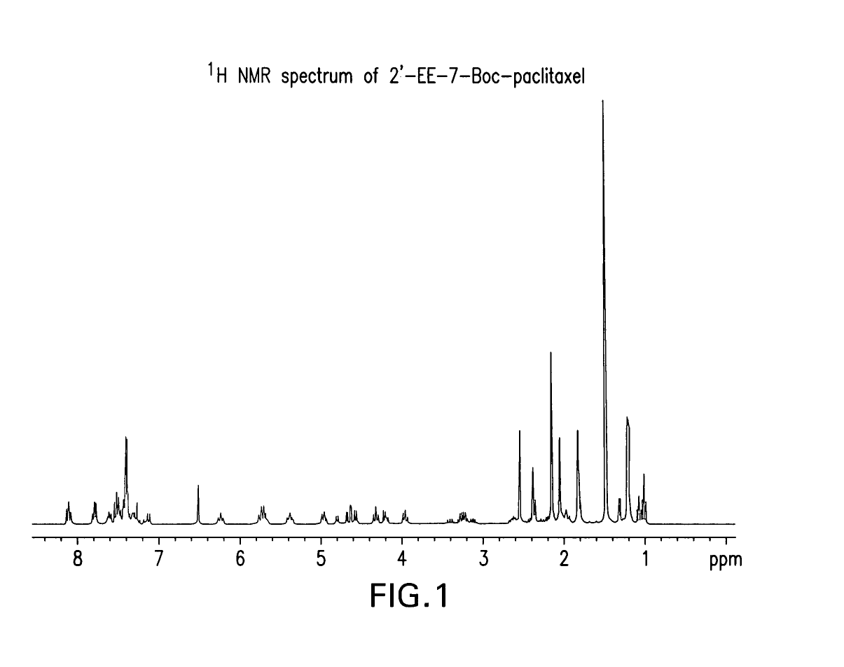

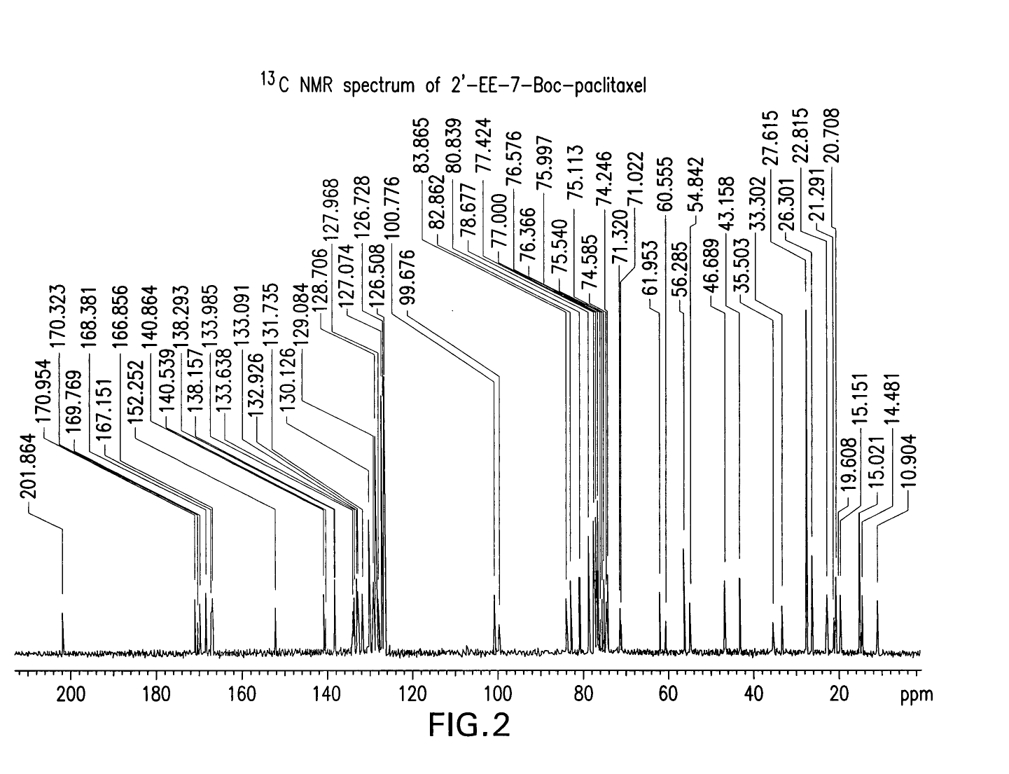

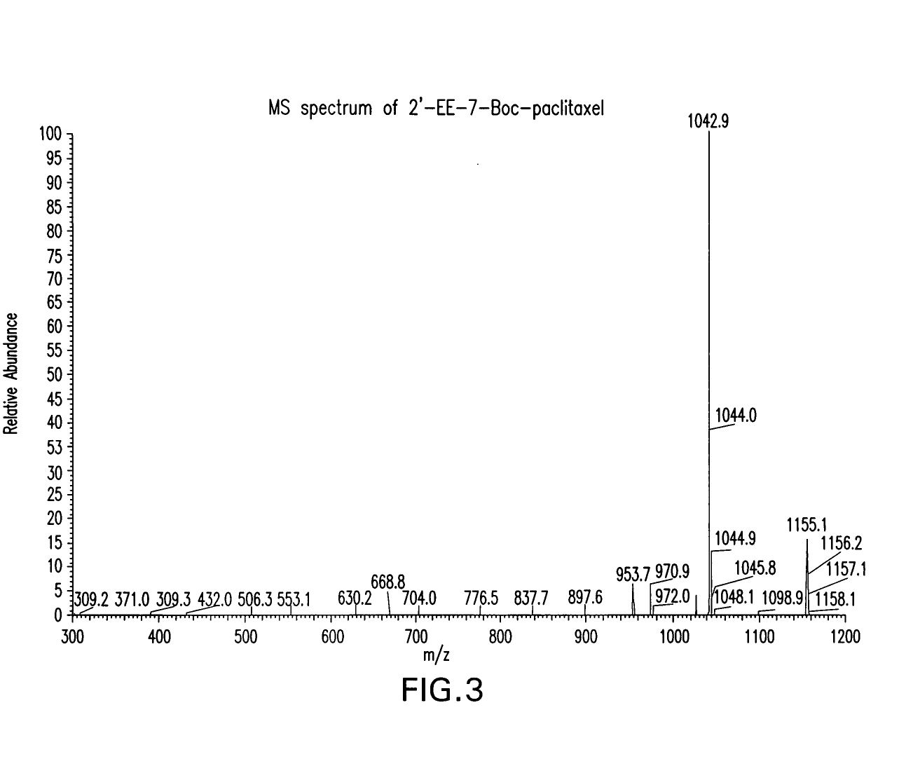

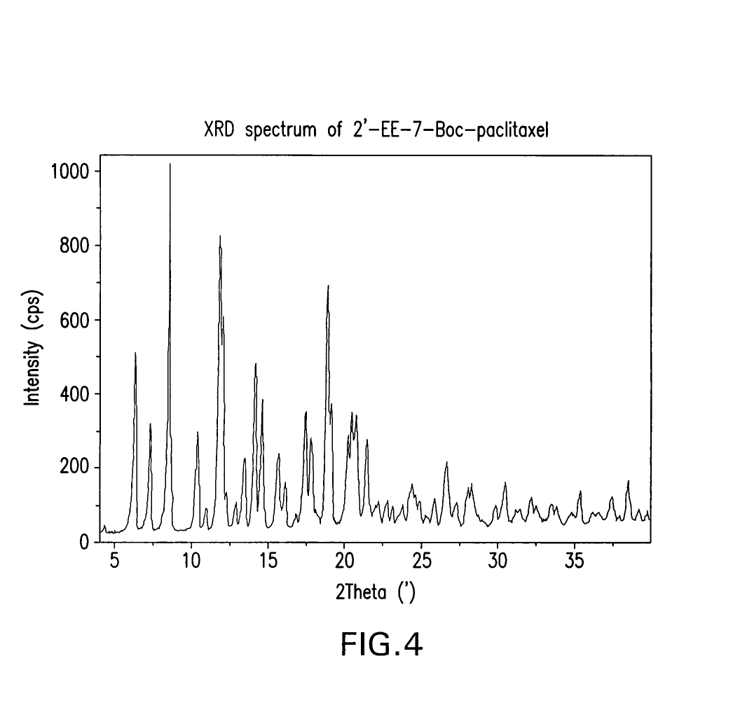

2′-EE-7-Boc paclitaxel is characterized by a ¹H NMR spectrum as shown in FIG. 1; a ¹³C NMR spectrum as shown in FIG. 2; a MS spectrum as shown in FIG. 3; or a PXRD spectrum as shown in FIG. 4.

2′-EE-7-Boc paclitaxel can be prepared according to a process comprising reacting 7-Boc-baccatin III of formula 2

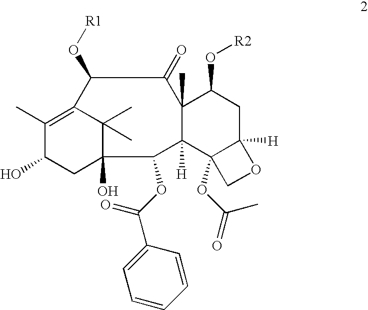

with a β-lactam of formula 3

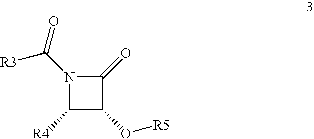

wherein R₁ is acetyl, R₂ is tert-butyloxycarbonyl (BOC), R₃ and R₄ are phenyl and R₅is 1-ethoxyethyl.

7-Boc-baccatin III can be prepared, for example, according to the process disclosed in International Patent Publication WO 2005/118563, or according to the procedure disclosed herein.

The process comprises reacting 13-acetyl-9-dihydro-baccatin III (9-DHB), an organic base and di-tert-butyl dicarbonate (BOC-anhydride) in a solvent.

Preferably, the solvent is selected from the group consisting of a C₃-C₅ ketone, a C₁-C₃ halogenated aliphatic hydrocarbon, a C₆-C₈ aromatic hydrocarbon, a C₃-C₄ alcohol, a C₂-C₃ nitrile, and mixtures thereof,

Preferably, the C₃-C₅ ketone is acetone or methyl ethylketone, the C₁-C₃ halogenated aliphatic hydrocarbon is dichloromethane (DCM), the C₆-C₈ aromatic hydrocarbon is toluene,

the C₃-C₄ alcohol is tert-butanol, the C₂-C₃ nitrile is acetonitrile. More preferably the solvent is DCM.

Preferably, the organic base is a tertiary amine, more preferably 4-dimethylaminopyridine (DMAP) or 4-pyrrolidino pyridine.

Preferably, the base is used in an amount of about 0.05 to about 1.0 mole equivalent to the used 9-DHB, more preferably from about 0.1 to about 0.2 mole equivalent.

The reaction with BOC-anhydride is preferably performed at a temperature of about −110° C. to about 40° C., more preferably from about −10° C. to about room temperature, most preferably at about 0° C.

Preferably, diatomaceous earth and a solvent are added to the said solution, and an aqueous solution of chromium (VI) oxide and diluted sulfuric acid is added dropwise. Preferably, the addition is performed at a temperature of about −5° C. to about room temperature, more preferably from about 0° C. to about 5° C., during a period of about one half-hour to about 2 hours, preferably about one hour.

Preferably, the solvent added with the diatomaceous earth is a C₂-C₃ nitrile or a C₃-C₅ ketone, more preferably acetonitrile or acetone, most preferably, acetonitrile.

The reaction is preferably monitored by HPLC. At the end of the reaction, the obtained solid is preferably removed by filtration. After filtering, the filtrate is diluted with a mixture of a C₁-C₃ alcohol, water and a water immiscible organic solvent to give two phases. Preferably, the C₁-C₃ alcohol is methanol. Preferably, the water immiscible organic solvent is a C₆-C₉ aromatic hydrocarbon or a C₁-C₃ halogenated aliphatic hydrocarbon, more preferably, the C₆-C₈ aromatic hydrocarbon is toluene and the C₁-C₃ halogenated aliphatic hydrocarbon is dichloromethane.

Next, the phases may be separated. The organic phase is preferably filtered through silica gel and the silica gel is further washed with a mixture of an aromatic hydrocarbon and a ketone, preferably in a ratio of about 4:1, respectively. Preferably, the mixture of aromatic hydrocarbon and ketone is a mixture of a C₆-C₈ aromatic hydrocarbon and a C₃-C₅ ketone, more preferably, a mixture of toluene and acetone.

The eluate may be evaporated, to give a crystalline suspension. The precipitated product may be filtered and dried to give crystalline 13-acetyl-7-Boc-baccatin III.

The isolated 13-acetyl-7-Boc-baccatin III may be further dissolved in a solvent and an aqueous solution of sodium borohydride may be added.

Preferably, the solvent is chosen from a list consisting of a C₄-C₆ ether, a C₁-C₅ alcohol, a C₂-C₃ nitrile and mixtures thereof. Preferably, the C₄-C₆ ether is tetrahydrofuran (THF) or 2-methyltetrahydrofuran. Preferably, the C₁-C₅ alcohol is methanol, ethanol, 1-propanol, 2-propanol or tert-butanol, Preferably, the C₂-C₃ nitrile is acetonitrile. Most preferably the solvent is THF.

Preferably, the reaction is performed at a temperature of about 0° C. to about room temperature, more preferably at about 0° C. to about 10° C.

The reaction is preferably monitored by HPLC. When the conversion of 13-acetyl-7-Boc-baccatin III is higher than about 70%, the reaction may be quenched by the addition of a water immiscible organic solvent and the phases may be separated.

Preferably, the water immiscible organic solvent is a C₁-C₃ halogenated aliphatic hydrocarbon or a C₆-C₉ aromatic hydrocarbon. Preferably, the C₁-C₃ halogenated aliphatic hydrocarbon is dichloro-methane. Preferably, the C₆-C₉ aromatic hydrocarbon is toluene. More preferably, the water immiscible organic solvent is toluene.

The separated organic phase may be concentrated and the residue may be crystallized from the water immiscible organic solvent, e.g., by cooling or by adding an antisolvent, obtaining 7-Boc-baccatin III which can be re-crystallized from the same solvent. The mother liquors can be subjected to chromatography and an additional crop of 7-Boc-baccatin III can be isolated. Also the unreacted 13-acetyl-7-Boc-baccatin III can be isolated and recycled.

7-Boc-baccatin III can be converted to 2′-EE-7-Boc paclitaxel via route A or via route B.

The process via route A comprises reacting 7-Boc-baccatin III of formula 2

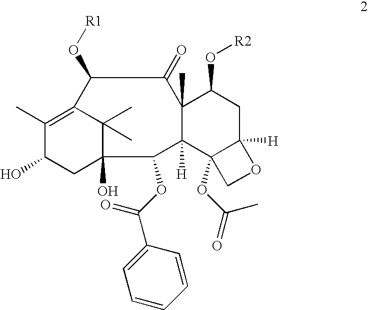

with EE-beta-lactam of formula 3

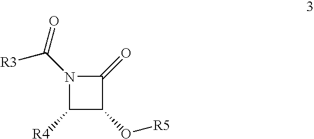

wherein R₁ is acetyl, R₂ is tert-butyloxycarbonyl (BOC), R₃ and R₄ are phenyl and R₅is 1-ethoxyethyl, in a solvent selected from a group consisting of an aromatic hydrocarbon, a halogenated aliphatic hydrocarbon, and mixtures thereof.

Preferably, the aromatic hydrocarbon is a C₆-C₈ aromatic hydrocarbon, more preferably, toluene or xylene, most preferably, toluene. Preferably, the halogenated aliphatic hydrocarbon is a C₁-C₃ halogenated aliphatic hydrocarbon, more preferably dichloromethane (DCM). More preferably, the solvent is toluene or DCM, most preferably toluene.

Preferably, 7-Boc-baccatin III, EE-β-lactam and the solvent are combined, preferably at room temperature, providing a suspension. Preferably, the β-lactam is present in the suspension in an amount of about 2 mole equivalent, about 2.5 mole equivalent or about 3 mole equivalent per mole equivalent of 7-Boc-baccatin III, as compared to about 5 mole equivalents that are used in the prior art per mole of protected baccatin. Since the β-lactam is an expensive reagent, it is advantageous to use it in smaller amounts.

Preferably, to this suspension is then added a catalytic amount of an organic base, providing a reaction mixture which is maintained at room temperature. Preferably, the organic base is a tertiary amine, more preferably 4-dimethyl-aminopyridine (DMAP), 4-pyrrolidino-pyridine and mixtures thereof, most preferably, the base is 4-pyrrolidino-pyridine.

Preferably, the amount of the base can be about 0.1 to about 0.5, about 0.2 to about 0.4, more preferably about 0.1, about 0.2, about 0.3, about 0.4 or about 0.5 mole equivalent per mole of 7-Boc-baccatin III compared to one mole equivalent used in the prior art procedure.

Typically, the reaction mixture is maintained at room temperature to allow the formation of 2′-EE-7-Boc paclitaxel. Preferably, the reaction mixture is maintained for about 12 to about 48 hours, about 16 to about 48 hours, about 16 to about 36 hours, or about 24 to about 36 hours, during which the conversion of 7-Boc-baccatin III of formula III to 2′-EE-7-Boc paclitaxel can be monitored. Ordinarily, the monitoring can be done by HPLC or TLC.

The obtained 2′-EE-7-Boc paclitaxel can then be recovered, for example by combining the reaction mixture with water and a water-immiscible organic solvent, providing a two-phase system, cooling the two-phase system to obtain a suspension comprising the product, and filtering the suspension to isolate the precipitated product in the form of crystals.

Preferably, the water-immiscible solvent is an aromatic hydrocarbon, more preferably, a C₆-C₈ aromatic hydrocarbon, most preferably, toluene.

The product can be isolated at about room temperature. Preferably, the two-phase system is cooled to a temperature below about 15° C., more preferably to about 5° C. The suspension can be filtered after the addition of the solvent. Preferably, the suspension is maintained before filtration for 4 hours, about 6 hours, about 8 hours, or about 12 hours, most preferably for about 8 hours.

The process via route B comprises reacting 7-Boc-baccatin III of formula 2

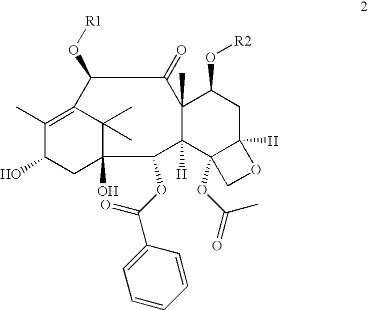

with EE-beta-lactam of formula 3

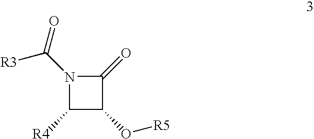

and a catalytic amount of a base, selected from the group consisting of an organometallic base, a metal hydride and mixtures thereof, providing a reaction mixture, and then quenching the reaction mixture, providing the intermediate 2′-EE-7-Boc paclitaxel, wherein R₁ is acetyl, R₂ is tert-butyloxycarbonyl (BOC), R₃ and R₄ are phenyl and R₅ is 1-ethoxyethyl.

Preferably, the reaction is done in the presence of an aprotic organic solvent. More preferably, the aprotic organic solvent is selected from the group consisting of: ether, an aromatic hydrocarbon, nitrile and mixtures thereof.

Preferably, the ether is a C₄-C₆ ether, more preferably, tetrahydrofuran (THF) or methyltetrahydrofuran, most preferably THF. Preferably, the aromatic hydrocarbon is a C₇-C₉ aromatic hydrocarbon, more preferably, toluene or xylene, most preferably, toluene. Most preferably, the organic solvent is a mixture of THF and toluene or a mixture of methyltetrahydrofuran and toluene, even more preferably, a mixture of THF and toluene.

7-Boc-baccatin III of formula 2 and the EE-beta-lactam of formula 3 may be first combined with the aprotic organic solvent to obtain a solution.

Preferably, about 1.2 to about 2.5 mole equivalent of the lactam of formula 3 per mole equivalent of the compound of formula 2 is present in the solution. More preferably about 1.3 mole equivalent of the lactam of formula 3 is used.

To this solution is then added the base. Suitable bases are organometallic bases or metal hydrides. Preferably, the organometallic base is selected from a group consisting of: lithium bis(trimethylsilyl) amide (LHMDS), sodium bis(trimethylsilyl) amide (NaHMDS), lithium diisopropylamide (LDA), butyllithium, methyllithium, and mixtures thereof. More preferably, the organometallic base is lithium bis(trimethylsilyl) amide. Preferably, the metal hydride is sodium hydride.

More preferably, the base is an organometallic base, more preferably, the organometallic base is lithium bis(trimethylsilyl) amide.

The base is added in a catalytic amount, although the common practice in such reactions is to use at least a stoichiometric amount of the base.

As used herein, the term “catalytic amount” in reference to the amount of the base corresponds to about 0.07 to about 0.4 mole equivalent per mole equivalent of the compound of the baccatin III of formula 2. In some embodiments, the amount of the base corresponds to about 0.07, about 0.08, about 0.09, about 0.1, about 0.2, about 0.3, or about 0.4 mole equivalents per mole equivalent of the compound of 7-Boc baccatin III of formula 2.

Preferably, the base is added in an amount of about 0.09 mole equivalent per mole equivalent of the compound of formula 2.

Typically, the base can be used neat (without a solvent) or in a solution wherein the solvent is an aprotic organic solvent. Suitable solvents are described above.

Preferably, the above reactants and solvent are combined at about room temperature, providing a reaction mixture. This reaction mixture is then maintained, preferably at a temperature of about 10° C. to about 50° C., more preferably at about room temperature.

According to common practice, side reactions, such as epimerization, can occur at such temperatures. However, these side reactions do not occur, or occur at a much reduced level, under the conditions of the current reaction. See, e.g., Example 1 herein, where the conversion of 7-Boc baccatin III to 2′EE-7-Boc paclitaxel is more than 99%, or Example 4 herein, where 2′EE-7-Boc paclitaxel is obtained at a purity level of 97.4%.

Preferably, the reaction mixture is maintained, preferably at room temperature, from about 1 to about 6 hours, about 2 to about 4 hours, about 1 hour, about 2 hours, about 3 hours, about 4 hours, about 5 hours, or about 6 hours. More preferably, the reaction mixture is maintained at room temperature for about 3 hours.

The reaction provides 2′-EE-7-Boc paclitaxel of formula 1. The reaction mixture containing 2′-EE-7-Boc paclitaxel may then be quenched and 2′-EE-7-Boc paclitaxel may be isolated. Typically, the quenching is performed by combining the reaction mixture with water.

The obtained 2′-EE-7-Boc paclitaxel can then be recovered, for example, by extracting 2′-EE-7-Boc paclitaxel from the quenched reaction mixture with an organic solvent, followed by filtering the obtained extract through a solid phase, removing the solvent and crystallizing 2′-EE-7-Boc paclitaxel. Preferably, the organic solvent is an aromatic hydrocarbon as described above, more preferably, toluene. Preferably, the solid phase is silica gel. Alternatively 2′-EE-7-Boc paclitaxel can crystallize directly from the quenched reaction mixture. Then 2′-EE-7-Boc paclitaxel is isolated by filtration. The obtained 2′-EE-7-Boc paclitaxel may be in the form of crystals, having a purity of at least 97% area by HPLC.

The obtained 2′-EE-7-Boc paclitaxel can be used to prepare paclitaxel, via a deprotection step (see scheme 1). In this step, formic acid alone or in combination with other organic acids is used, thus forming paclitaxel within 1-5 hours without formation of degradation products. Furthermore, the obtained paclitaxel readily crystallizes from the reaction mixture, thus simplifying the isolation process, and the paclitaxel is obtained in higher yields than those reported previously.

The preparation can be done by a process comprising reacting 2′-EE-7-Boc paclitaxel with formic acid or with a mixture of formic acid and a second organic acid in the presence or absence of a solvent which is immiscible with formic acid or with the mixture of formic acid and the second organic acid.

Preferably, the reaction of 2′-EE-7-Boc paclitaxel with formic acid or with a mixture of formic acid and a second organic acid is done in the absence of a solvent. When the reaction is done in the presence of a solvent, the solvent excludes THF and is preferably an aliphatic hydrocarbon, more preferably, a C₅-C₈ aliphatic hydrocarbon, most preferably, hexane.

Preferably, the second organic acid is a C₂-C₃ organic acid, more preferably acetic or propionic acid, most preferably acetic acid.

Preferably, the reaction is performed at a temperature of about 0° C. to about 10° C., more preferably about 0° C. to about 5° C., most preferably about 0° C. to about 2° C.

The progress of the reaction can be monitored by following the disappearance of the starting material 2′-EE-Boc paclitaxel, typically by methods such as HPLC and TLC.

The obtained paclitaxel can then be recovered, for example, by combining the reaction mixture with water and a water-immiscible organic solvent, providing a two-phase system, cooling the two-phase system to obtain a suspension comprising the product, and filtering the suspension to isolate the precipitated crude product in the form of crystals.

Preferably, the water-immiscible organic solvent is a C₆-C₈ aromatic hydrocarbon, more preferably toluene.

Preferably, the two-phase system is cooled to a temperature of about 0° C. to about 8° C., most preferably about 0° C. to about 5° C. The cooling usually provides a suspension, which can be further maintained at such temperature to increase the yield of the precipitated product. Preferably, the suspension is maintained for about 1 hour.

Preferably, the obtained crude paclitaxel has a purity of at least about 85%, at least about 87%, at least about 90% or at least about 95% area by HPLC, preferably, about 85% to about 97%, about 87% to about 95%, about 90% to about 95%, or about 90% to about 93% area by HPLC. The obtained crude paclitaxel can contain about 1%, about 2%, about 3%, about 4%, about 5%, or about 6% or less area by HPLC of 7-Boc paclitaxel and a total amount of about 1%, about 2%, about 3%, about 4%, or about 5% or less of other impurities (area by HPLC).

The obtained crude paclitaxel can be purified by a process comprising crystallizing paclitaxel from a mixture comprising a C₃-C₅ ketone, a C₆-C₉ aromatic hydrocarbon and water, preferably the mixture is a mixture of acetone, toluene and water.

Typically, the crystallization comprises providing a solution of paclitaxel in a mixture of the C₃-C₅ ketone and the C₆-C₈ aromatic hydrocarbon, and combining the solution with a mixture of water and the C₆-C₉ aromatic hydrocarbon to obtain a suspension.

The precipitated paclitaxel can then be recovered from the suspension, for example by filtering the suspension.

Preferably, the obtained purified paclitaxel has a purity of at least about 95%, at least about 96%, at least about 97%, at least about 98% or at least about 99% area by HPLC. In some embodiments, the obtained purified paclitaxel has a purity of about 95%, about 96%, about 97%, about 98%, about 99%, or about 100% area by HPLC. The purified paclitaxel can contain about 0.5% or less, about 1% or less, about 2% or less, or about 3% or less by area HPLC of 7-Boc paclitaxel and a total amount of about 0.5% or less, about 1% or less, about 2% or less, or about 3% or less of other impurities. In some embodiments, the purified paclitaxel can contain about 0%, about 0.5%, about 1%, about 2%, or about 3% by area HPLC of 7-Boc paclitaxel and a total amount of about 0%, about 0.5%, about 1%, about 2%, or about 3% of other impurities. In some embodiments, the obtained purified paclitaxel has a purity of about 96% or about 99.5% (by area HPLC). In some embodiments, the purified paclitaxel can contain about 1.5% by area HPLC of 7-Boc paclitaxel and a total amount of about 1.9% or about 0.5% of other impurities by area HPLC.

The process of route B can be used in order to prepare also other intermediates of taxanes of formula 1

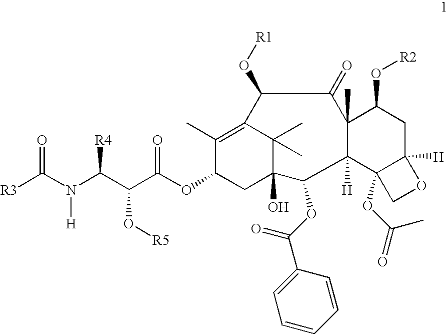

The taxane intermediate preparation comprises reacting a 7-protected derivative of baccatin or 7,10-diprotected derivative of 10-deacetyl-baccatin (10-DAB) of formula 2

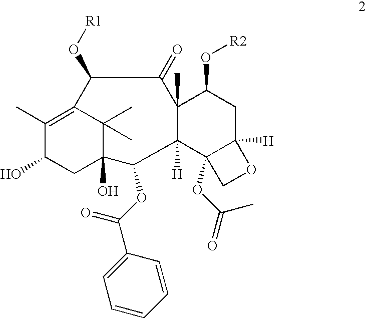

with a β-lactam of formula 3

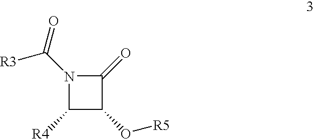

and a catalytic amount of a base, selected from the group consisting of an organometallic base, a metal hydride and mixtures thereof, providing a reaction mixture, and then optionally quenching the reaction mixture, providing the intermediate of formula 1, wherein

R₁, R₂ and R₅ are independently a hydroxyl protecting group;

R₃ is phenyl, substituted phenyl, a straight or branched alkyl, alkenyl, cycloalkyl, cycloalkenyl or an R₆—O— group in which R₆ is

phenyl, substituted phenyl group, a straight or branched alkyl, a straight or branched alkenyl group, a straight or branched alkynyl, a cycloalkyl, a cycloalkenyl, a bicycloalkyl substituent, or a saturated or unsaturated nitrogen;

and

R₄ is phenyl or a substituted phenyl group.

Preferably, R₁ is acetyl.

Preferably R₂ and R₅ are different. Preferably, R₂ is tert-butyloxycarbonyl (BOC). Preferably, R₅ is ethoxyethyl, more preferably 1-ethoxyethyl.

Preferably, R₃ is phenyl, a substituted phenyl (substituted by chloro, bromo, fluoro, and C₁-C₆ straight or branched chain alkyl), a C₁-C₁₂ straight or branched alkyl, a C₂-C₁₂ alkenyl, a C₄-C₁₅ cycloalkyl, a C₄-C₁₅ cycloalkenyl or an R₆—O— group in which R₆ is

preferably phenyl, substituted phenyl group, a C₁-C₈ straight or branched alkyl, a C₂-C₈ straight or branched alkenyl group, a C₃-C₈ straight or branched alkynyl, a C₃-C₇ cycloalkyl, C₄-C₇ cycloalkenyl, a C₇-C₁₁ bicycloalkyl substituent, or a saturated or unsaturated nitrogen.

More preferably, R₃ is phenyl or tert-butyloxy.

Preferably, R₄ is a phenyl or a phenyl group substituted by chloro, bromo, fluoro, and C₁-C₆ straight or branched chain alkyl. Most preferably, R₄ is phenyl.

Preferably, when R₆ is a substituted phenyl group it is substituted by chloro, bromo, fluoro, and C₁-C₆ straight or branched chain alkyl.

When R₁ is acetyl and R₂ is BOC, R₃ is tert-butyloxy, R₄ is phenyl and R₅ is 1-ethoxyethyl, the compound of formula 1 refers to 10-acetyl-7-Boc-2′-(1-ethoxyethyl)-docetaxel (10-Ac-2′-EE-7-Boc docetaxel) of the following formula

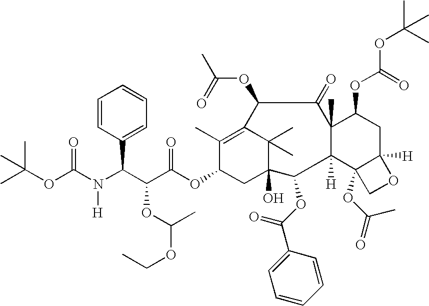

When R₁ and R₂ are BOC, R₃ is tert-butyloxy, R₄ is phenyl and R₅ is 1-ethoxyethyl, the compound of formula 1 refers to 7,10-diBoc-2′-(1-ethoxyethyl)-docetaxel (2′-EE-7,10-diBoc docetaxel) of the following formula

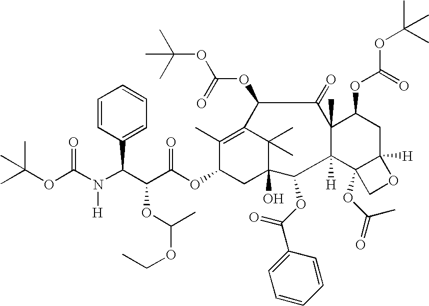

When R₁ is acetyl and R₂ is BOC, the compound of formula 2 refers to 7-Boc-baccatin of the following formula

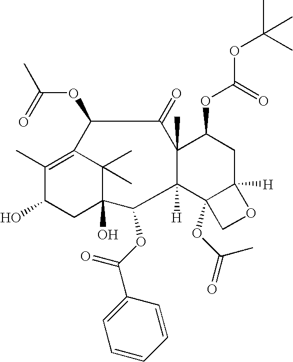

When R₁ and R₂ are BOC, the compound of formula 2 refers to 7,10-diBoc-baccatin of the following formula

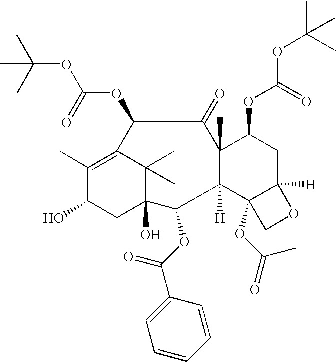

When R₃ is phenyl, R₄ is phenyl and R₅ is 1-ethoxyethyl, the compound of formula 3 refers to an EE-beta-lactam of the following formula

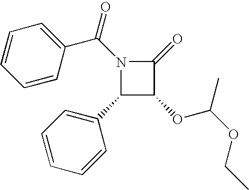

When R₃ is tert-butyloxy, R₄ is phenyl and R₅ is 1-ethoxyethyl, the compound of formula 3 refers to an EE-beta-lactam of the following formula

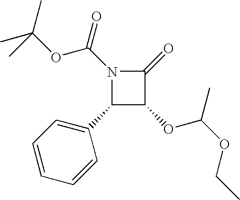

The obtained compound of formula 1 can then be used to prepare taxanes such as paclitaxel or docetaxel. The conversion of the compound of formula 1 to taxane is conducted by removing the hydroxyl protecting groups from positions 7, 10 and 2′. When paclitaxel is prepared, the R₂ and R₅ protecting groups are removed. When docetaxel is prepared, the R₁, R₂ and R₅ are removed.

The removal of the protecting groups can be conducted, for example, according to methods known in the art or by the process described herein (and see Examples 7-9), as described for 2′-EE-7-Boc paclitaxel.

### EXAMPLES

### HPLC Method

Examples 6, 7, 8, 9, 10 and 11 were analyzed according to the USP Monograph for semisynthetic paclitaxel. The other examples were analyzed according to the following method.

| Column: | Reversed phase, 1.8 μm, 100 × 4.6 mm | | |
|---|---|---|---|
| Column temperature: | 40° C. | | |
| Mobile phase A: | water-acetonitrile 3:2 (v/v) | | |
| Mobile phase B: | acetonitrile | | |
| Flow rate: | 1.2 ml/min | | |
| Injection: | 5 μl | | |
| Detection: | UV at 230 nm | | |
| Gradient: | time | % MPA | % MPB |
| | 0 | 90 | 10 |
| | 1 | 90 | 10 |
| | 5 | 58 | 42 |
| | 8 | 10 | 90 |
| | 10 | 10 | 90 |
| | 13 | 90 | 10 |

### Example 1

### Preparation of 2′-EE-7-Boc Paclitaxel

To a mixture of 7-Boc-baccatin III (98% purity, 15.35 g, 22.38 mmol) and (3R,4S)-3-(1-ethoxyethoxy)-4-phenyl-N-benzoyl-2-azetidinone (9.81 g, 28.94 mmol, 1.29 moleq.) in tetrahydrofuran (60 ml) was slowly added 1 M THF solution of lithium bis(trimethylsilyl) amide (2 ml, 2.0 mmol, 0.09 moleq) at room temperature. The solution was stirred at room temperature for 3 h. Reaction mixture was then partitioned between toluene (30 ml) and water (30 ml). The organic layer was separated and the aqueous layer was extracted with toluene. Organic phases were combined, filtered through silica gel (15 g) and concentrated, obtaining a crystalline product. 19.7 g of crystalline 2′-EE-7-Boc paclitaxel (97.3% purity) was isolated. The conversion of 7-Boc-baccatin III was more than 99%.

### Example 2

### Preparation of 2′-EE-7-Boc Paclitaxel

8.4 g of (3R,4S)-3-(1-ethoxyethoxy)-4-phenyl-N-benzoyl-2-azetidinone was placed into a bulb and dissolved in 8.4 ml toluene. 5.0 g of 7-Boc-baccatin III (purity 98.3%) and 0.5 g of DMAP were added and the mixture was stirred at room temperature. After 24 hours the conversion of 7-Boc-baccatin III was about 85%. Therefore the stirring was prolonged for another 24 hours, when the conversion was more than 97%. Then the mixture was diluted with 50 ml toluene and 25 ml water and stirred for 8 hours at about 5° C. After that time the suspension was filtered and washed with 5 ml toluene, obtaining 6.4 g of 2′EE-7-Boc paclitaxel having a purity of more than 97% according to HPLC.

### Example 3

### Preparation of 2′-EE-7-Boc Paclitaxel

9.1 g of (3R,4S)-3-(1-ethoxyethoxy)-4-phenyl-N-benzoyl-2-azetidinone was placed into a bulb and dissolved in 9.1 ml toluene. 5.5 g of 7-Boc-baccatin III (purity 98.3%) and 0.5 g of 4-pyrrolidino-pyridine were added and the mixture was stirred at room temperature. After 24 hours the conversion of 7-Boc-baccatin III was about 96%. The mixture was diluted with 50 ml toluene and 25 ml water and stirred for 8 hours at about 5° C. After that time the suspension was filtered and washed with 5 ml toluene, obtaining 6.8 g of 2′EE-7-Boc paclitaxel having a purity of more than 97% according to HPLC.

### Example 4

### Preparation of 2′-EE-7-Boc Paclitaxel

To a mixture of 7-Boc-baccatin (98% purity, 15.70 g, 22.89 mmol) and (3R,4S)-3-(1-ethoxyethoxy)-4-phenyl-N-benzoyl-2-azetidinone (9.7 g, 28.62 mmol, 1.25 moleq.) in tetrahydrofuran (30 ml) and toluene (30 ml) was slowly added 1 M THF solution of lithium bis(trimethylsilyl) amide (2.5 ml, 2.5 mmol, 0.11 moleq) at room temperature. The mixture was stirred at room temperature 2.5 h. Reaction mixture was then diluted with toluene (100 ml) and aqueous solution of acetic acid (1 ml in 30 ml of water) and then with 200 ml hexane and the suspension was stirred for 30 minutes at room temperature. The crystalline product was separated by filtration and re-crystallized from toluene, obtaining crystalline 2′-EE-7-Boc paclitaxel (20.2 g, 97.4% purity).

### Example 5

### Coupling 7-Boc-baccatin III with (3R,4S)-3-(1-ethoxyethoxy)-4-phenyl-N-benzoyl-2-azetidinone using dichloromethane and 4-pyrrolidinopyridine

9.0 g of (3R,4S)-3-(1-ethoxyethoxy)-4-phenyl-N-benzoyl-2-azetidinone was placed into a bulb and dissolved in 9.0 ml dichloromethane. 5.5 g of 7-Boc-baccatin III (purity 98.3%) and 0.5 g of 4-pyrrolidino-pyridine were added and the mixture was stirred at room temperature. After 48 hours the conversion of 7-Boc-baccatin III was about 98%. The mixture was diluted with 50 ml toluene and 25 ml water and stirred for 8 hours at about 5° C. After that time, the suspension was filtered and washed with 5 ml toluene, obtaining 6.6 g of 2′EE-7-Boc paclitaxel having a purity of more than 97% according to HPLC.

### Comparative Example 6

### Coupling 7-Boc-baccatin III with (3R,4S)-3-(1-ethoxyethoxy)-4-phenyl-N-benzoyl-2-azetidinone according to U.S. Pat. No. 5,175,315, Example 2

Example 2 of U.S. Pat. No. 5,175,315 describes using 7-O-triethylsilyl-baccatin III instead of 7-Boc baccatin III.

109 mg (0.320 mmol) of (3R,4S)-3-(1-ethoxyethoxy)-4-phenyl-N-benzoyl-2-azetidinone, 45 mg (0.064 mmol) of 7-O-triethylsilyl-baccatin III, 7.8 mg (0.064 mmol) of DMAP and 0.032 ml pyridine is the loading according to this example.

Based on that, the following analogous experiment was performed:

1.2 g of (3R,4S)-3-(1-ethoxyethoxy)-4-phenyl-N-benzoyl-2-azetidinone, 0.5 g of 7-Boc-baccatin III, 0.1 g of DMAP and 0.4 ml pyridine were placed in a bulb, obtaining very viscous mixture which was impossible to stir by mechanical stirrer. The bulb was held under room temperature and stirred from time to time by a spatula. After 24 hours, the conversion of 7-Boc-baccatin III was only about 50% and after 3 days the conversion was about 85%. Then the mixture was dissolved in ethyl acetate and the solution was washed with water, concentrated and the residue was purified by chromatography, obtaining 2-EE-7-Boc paclitaxel.

### Example 7

### Preparation of Paclitaxel from 2′-EE-7-Boc Paclitaxel

20.0 g of 2′EE-7-Boc paclitaxel having a purity of more than 97% was added under stirring to 100 ml formic acid (reagent grade) cooled to about 8° C. After 1 hour, the conversion to paclitaxel was more than 90% and thus 100 ml of toluene and 300 ml water were added and stirring at about 8° C. was prolonged for another 1 hour. Then the crystalline product was separated by filtration and the cake was washed with water and toluene, obtaining 14.5 g of crude paclitaxel, containing 91.7% paclitaxel, 3.9% 7-Boc paclitaxel and 4.4% of the sum of the other impurities (analyzed by the HPLC method described in the USP Monograph for semisynthetic paclitaxel).

### Example 8

### Preparation of Paclitaxel from 2′-EE-7-Boc Paclitaxel

20.0 g of 2′EE-7-Boc paclitaxel having a purity of more than 97% was added under stirring to 100 ml formic acid (reagent grade) and 15 ml of acetic acid, cooled to about 0° C. After 2 hours, the conversion to paclitaxel was more than 90% and thus 100 ml of toluene and 300 ml water were added and stirring at about 5° C. was prolonged for another 1 hour. Then the crystalline product was separated by filtration and the cake was washed with water and toluene, obtaining 15.2 g of crude paclitaxel, containing 92.3% paclitaxel, 4.3% 7-Boc paclitaxel and 3.4% of the sum of the other impurities (analyzed by the HPLC method described in the USP Monograph for semisynthetic paclitaxel).

### Example 9

### Preparation of Paclitaxel from 2′-EE-7-Boc Paclitaxel

20.0 g of 2′EE-7-Boc paclitaxel having a purity of more than 97% was suspended in 100 ml n-hexane and the suspension was stirred at about 0° C. A mixture of 100 ml of formic acid (reagent grade) and 15 ml of acetic acid was added within about 15 minutes to the stirred suspension of 2′EE-7-Boc paclitaxel and the stirring was prolonged for another 2.5 hours, when the conversion to paclitaxel was more than 90% and thus 100 ml of toluene and 300 ml water were added and stirring at about 5° C. was prolonged for another 1 hour. Then the crystalline product was separated by filtration and the cake was washed with water and toluene, obtaining 15.9 g of crude paclitaxel, containing 92.4% paclitaxel, 4.5% 7-Boc paclitaxel and 3.1% of the sum of the other impurities (analyzed by the HPLC method described in the USP Monograph for paclitaxel).

### Example 10

### Purification of Crude Paclitaxel

15.0 g of crude paclitaxel containing 92.4% paclitaxel, 4.5% 7-Boc paclitaxel and 3.1% of the sum of the other impurities was dissolved in a mixture of 50 ml acetone and 50 ml toluene and the solution was brought to crystallization by addition 3 ml of water and then 250 ml toluene. After 2 hours stirring at room temperature, the crystalline paclitaxel was separated and washed with toluene, obtaining 12.9 g of purified paclitaxel, containing 96.0% paclitaxel, 1.5% of 7-Boc paclitaxel and 1.9% of the sum of the other impurities (analyzed by the HPLC method described in the USP Monograph for semisynthetic paclitaxel).

### Example 11

### Purification of Paclitaxel

25.0 g of paclitaxel containing 98.9% paclitaxel and 1.1% of the sum of the other impurities was dissolved in a mixture of 75 ml acetone and 75 ml toluene and the solution was brought to crystallization by addition 4 ml of water and then 270 ml toluene. After 2 hours stirring at room temperature, the crystalline paclitaxel was separated and washed with toluene, obtaining 23.7 g of purified paclitaxel, containing 99.5% paclitaxel, and 0.5% of the sum of the other impurities (analyzed by the HPLC method described in the USP Monograph for semisynthetic paclitaxel).

### Example 12

### Preparation of 7-Boc-13-acetyl-baccatin III

20 g of 9-DHB and 0.4 g of dimethylaminopyridine were suspended in 100 ml dichloromethane and the suspension was cooled to −5° C. 10 g of di-tert-butyl dicarbonate in 20 ml dichloro methane was added during one hour and the obtained solution was stirred at the temperature 0° C. for another for 1 hour. Then 100 ml acetonitrile and 20 g of diatomaceous earth was added and the temperature was held at 0° C. A solution of 5.6 g of chromium (VI) oxide in 3.5 mol water was added during 5 minutes and then the solution of 4.5 ml sulfuric acid in 20 ml acetonitrile was dropped within 2 hours. After finishing the reaction (HPLC monitoring) the solid part was filtered off and the cake was washed with 100 ml methanol. The filtrate was diluted with 50 ml dichloromethane and 100 ml water. The obtained phases were separated and the aqueous phase was twice more extracted with 20 ml dichloromethane. The organic extracts were joined and diluted with 100 ml toluene then filtered through 20 g silica gel. The silica gel was washed with a mixture of toluene and acetone (4:1) and the eluate was evaporated, obtaining a crystalline suspension. The product was filtered off and dried, obtaining 17.9 g of crystalline 7-Boc-13-acetyl-baccatin III with purity 97.4%. The mother liquors were purified by column chromatography and 2.3 g of crystalline product was obtained (purity 97.9%).

### Example 13

### Preparation of 7-Boc-baccatin III

20 g of the product from example 12 was dissolved in 450 ml tetrahydrofuran and the solution was cooled down to 0° C. A cool solution of sodium borohydride (7.0 g) in 150 ml of water was added and the solution was stirred under cooling in the range 0-4° C. for 7 hours under HPLC monitoring. When the conversion was more than 75% (after 7 hours), 150 ml toluene was added and the formed phases were separated. The aqueous phase was extracted twice more with 50 ml toluene and the combined toluene extracts were concentrated to obtain a crystalline suspension. The crystalline product was filtered off and washed. Then the crystalline product was dissolved in acetone (100 ml) and the solution was diluted with toluene (200 ml). The solution was filtered through 20 g silica gel and the effluent was concentrated to obtain a crystalline suspension. 11.1 g of 7-Boc-baccatin III (97.5% purity) was obtained after filtration and drying. The mother liquors from both crystallization steps were purified by column chromatography to obtain 2.9 g of 7-Boc-baccatin III (purity 96.7%) and 4.9 g of 7-Boc-13-acetyl-baccatin III (purity 78.2%).

### Example 14

### Preparation of Protected Paclitaxel According to U.S. Pat. No. 5,274,124, Example 7

To a solution of 10.0 g of 7-TES-baccatin (14.3 mmol) in 100 ml THF at −45° C. was added 14.3 ml of 1 M solution of lithium bis(trimethylsilyl) amide (14.3 mmol, 1.0 moleq). After 1 hour at −45° C., a solution of (3R,4S)-3-(triethylsilyloxy)-4-phenyl-N-benzoyl-2-azetidinone (8.2 g, 21.5 mmol, 1.50 moleq) in 100 ml THF was added and the solution was warmed to 0° C. The solution was partitioned between saturated aqueous sodium hydrogen carbonate and ethyl acetate-hexane (60:40). The organic phase was evaporated and the residue was purified by chromatography, followed by crystallization to obtain 2′,7-di-TES-paclitaxel (15.1 g).

### Example 15

### Preparation of Protected Docetaxel According to U.S. Pat. No. 6,187,916, Example 55

To a solution of 9.0 g (10 mmol) of 7,10-diTroc-10-deacetylbaccatin and 4.7 g (14 mmol) of (3R,4S)-3-(1-ethoxyethoxy)-4-phenyl-N-Boc-2-azetidinone in 500 ml of THF, was added at −30° C. 11 ml 1 M solution of sodium bis(trimethylsilyl) amide (11 mmol, 1.1 moleq.). After the reaction was complete, brine and dichloromethane were added and the phases were separated. The aqueous layer was extracted with dichloromethane. The combined organic layers were washed with brine, dried over sodium sulfate and concentrated. The crude oil was purified by chromatography on silica gel using ethyl acetate-hexanes (1/2) to give 11.7 g of the coupling product 2′-EE-7,10-ditroc-docetaxel as a white solid.

### Example 16

### Preparation of 2′-TES-7-Boc Paclitaxel

To a mixture of 7-Boc-baccatin (98% purity, 6.65 g, 9.70 mmol) and (3R,4S)-3-(triethylsilyloxy)-4-phenyl-N-benzoyl-2-azetidinone (4.45 g, 11.68 mmol, 1.24 moleq.) in tetrahydrofuran (15 ml) and toluene (15 ml) was slowly added 1 M THF solution of lithium bis(trimethylsilyl) amide (1.5 ml, 1.5 mmol, 0.15 moleq) at room temperature. The solution was stirred at room temperature for 2 h. The reaction mixture was then partitioned between toluene (45 ml) and an aqueous solution of acetic acid (0.1 ml in 15 ml of water). The resulting two phase liquid mixture was diluted with hexane (90 ml), which caused crystallization of product. The mixture was stirred 2.5 hours at room temperature and then filtered, obtaining crystalline 2′-TES-7-Boc paclitaxel (6.86 g, 95.0% purity).

### Example 17

### Preparation of 2′-EE-10-Acetyl-7-Boc-docetaxel

To a mixture of 7-Boc-baccatin (98% purity, 2.0 g, 2.90 mmol) and (3R,4S)-3-(1-ethoxyethoxy)-4-phenyl-N-Boc-2-azetidinone (1.3 g, 3.9 mmol) in tetrahydrofuran (8 ml) was slowly added 1 M THF solution of lithium bis(trimethylsilyl) amide (1 ml, 1 mmol) at room temperature. The solution was stirred at room temperature for 2 h. The reaction mixture was then partitioned between toluene (5 ml) and an aqueous solution of acetic acid (0.1 ml in 5 ml of water). The phases were separated and the aqueous phase was twice more extracted with toluene. The combined toluene extracts were concentrated and crystallized from toluene, to obtain crystalline 2′-EE-10-Acetyl-7-Boc docetaxel (1.8 g, 98.1% purity).

### Example 18

### Preparation of 2′-EE-7,10-Di-Boc Paclitaxel

To a mixture of 7,10-di-Boc-baccatin (1.0 g, 1.3 mmol) and (3R,4S)-3-(1-ethoxyethoxy)-4-phenyl-N-benzoyl-2-azetidinone (0.6 g, 1.8 mmol) in tetrahydrofuran (10 ml) was slowly added 1 M THF solution of lithium bis(trimethylsilyl) amide (0.5 ml, 0.5 mmol) at room temperature. The solution was stirred at room temperature for 2 h. The reaction mixture was then partitioned between toluene (30 ml) and an aqueous solution of acetic acid (0.1 ml in 10 ml of water). The phases were separated and the aqueous phase was twice more extracted with toluene. The combined toluene extracts were concentrated and crystallized from toluene, to obtain crystalline 2′-EE-7,10-di-Boc paclitaxel (0.9 g, 95.4% purity).

### Example 19

### Preparation of 2′-EE-7,10-di-Boc Docetaxel

To a mixture of 7,10-di-Boc-baccatin (1.5 g, 2.0 mmol) and (3R,4S)-3-(1-ethoxyethoxy)-4-phenyl-N-Boc-2-azetidinone (1.0 g, 3.0 mmol) in tetrahydrofuran (15 ml) was slowly added 1 M THF solution of lithium bis(trimethylsilyl) amide (1 ml, 1 mmol) at room temperature. The solution was stirred at room temperature for 2 h. The reaction mixture was then partitioned between toluene (50 ml) and an aqueous solution of acetic acid (0.1 ml in 10 ml of water). The phases were separated and the aqueous phase was twice more extracted with toluene. The combined toluene extracts were concentrated and crystallized from toluene, to obtain crystalline 2′-EE-7,10-di-Boc paclitaxel (1.1 g, 97.5% purity).

What is claimed is:

1. 7-Boc-2′-(1-ethoxyethyl)-paclitaxel, (2′-EE-7-Boc paclitaxel), of formula 1: 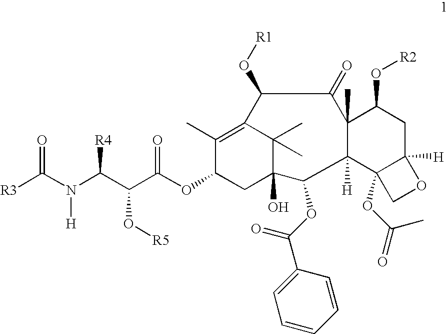
wherein R₁ is acetyl, R₂ is tert-butyloxycarbonyl (BOC), R₃ and R₄ are phenyl and R₅is 1-ethoxyethyl.

2. The compound of claim 1, wherein the compound is isolated.

3. The compound of claim 2, wherein the compound is solid.

4. The compound of claim 3, wherein the compound is crystalline.

5. A process for preparing the compound of claim 1 comprising reacting 7-Boc-baccatin III of formula 2 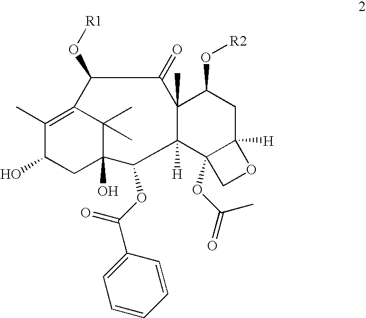
with EE-β-lactam of formula 3 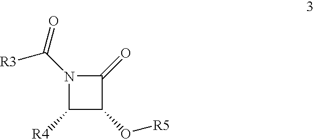
wherein R₁ is acetyl, R₂ is tert-butyloxycarbonyl (BOC), R₃ and R₄ are phenyl and R₅is 1-ethoxyethyl.

6. The process of claim 5 comprising
A) reacting 7-Boc-baccatin III of formula 2 with EE-beta-lactam of formula 3 in a solvent selected from a group consisting of an aromatic hydrocarbon, a halogenated aliphatic hydrocarbon, and mixtures thereof, or
B) reacting 7-Boc-baccatin III of formula 2 with EE-beta-lactam of formula 3 and a catalytic amount of a base selected from the group consisting of an organometallic base, a metal hydride and mixtures thereof, providing a reaction mixture, and then quenching the reaction mixture, providing 2′-EE-7-Boc paclitaxel of formula 1.

7. The process of claim 6 wherein route A) is carried out and wherein the aromatic hydrocarbon is a C₆-C₉ aromatic hydrocarbon and the halogenated aliphatic hydrocarbon is C₁-C₃ halogenated aliphatic hydrocarbon.

8. The process of claim 7 wherein the aromatic hydrocarbon is toluene or xylene, and the C₁-C₃ halogenated aliphatic hydrocarbon is dichloromethane (DCM).

9. The process of claim 6 wherein route A) is carried out and wherein the solvent comprises a catalytic amount of an organic base.

10. The process of claim 9, wherein the organic base is a tertiary amine.

11. The process of claim 10, wherein the base is selected from the group consisting of 4-dimethyl-aminopyridine (DMAP), 4-pyrrolidino-pyridine and mixtures thereof.

12. The process of claim 11 wherein the obtained 2′-EE-7-Boc paclitaxel is recovered.

13. The process of claim 6 wherein route B) is carried out and wherein the reaction is conducted in the presence of an aprotic organic solvent.

14. The process of claim 13, wherein the aprotic organic solvent is selected from the group consisting of: an ether, an aromatic hydrocarbon, a nitrile and mixtures thereof.

15. The process of claim 14 wherein the ether is a C₄-C₆ ether and the aromatic hydrocarbon is a C₇-C₉ aromatic hydrocarbon.

16. The process of claim 15 wherein the C₄-C₆ ether is tetrahydrofuran (THF) or methyltetrahydrofuran and the C₇-C₉ aromatic hydrocarbon is toluene or xylene.

17. The process of claim 6 wherein route B) is carried out and wherein about 1.2 to about 2.5 mole equivalent of the beta-lactam of formula 3 per mole equivalent of 7-Boc baccatin III of formula 2 is reacted with the compound of formula 2.

18. The process of claim 6 wherein route B) is carried out and wherein the organometallic base is selected from the group consisting of: lithium bis(trimethylsilyl) amide (LHMDS), sodium bis(trimethylsilyl) amide (NaHMDS), lithium diisopropylamide (LDA), butyllithium, methyllithium, and mixtures thereof and the metal hydride is sodium hydride.

19. The process of claim 18 wherein the base is present in a catalytic amount.

20. The process of claim 6 wherein route B) is carried out and wherein the reaction is performed at a temperature of about 110° C. to about 50° C.

21. The process of claim 6 wherein route B) is carried out and wherein the obtained 2′-EE-7-Boc paclitaxel is recovered.

22. A process for preparing paclitaxel comprising preparing 2′-EE-7-Boc paclitaxel according to the process of claim 6 and converting 2′-EE-7-Boc paclitaxel to paclitaxel.

23. The process of claim 22 wherein the conversion comprises reacting 2′-EE-7-Boc paclitaxel with formic acid or with a mixture of formic acid and a second organic acid in the presence or absence of a solvent wherein the solvent is immiscible in formic acid or in the mixture of formic acid and the second organic acid.

24. The process of claim 23, wherein the solvent is an aliphatic hydrocarbon.

25. The process of claim 24 wherein the aliphatic hydrocarbon is a C₅-C₈ aliphatic hydrocarbon.

26. The process of claim 25 wherein the C₅-C₈ aliphatic hydrocarbon is hexane.

27. The process of claim 26 wherein the second organic acid is a C₂-C₃ organic acid.

28. The process of claim 27 wherein the C₂-C₃ organic acid is acetic or propionic acid.

29. The process of claim 28 wherein the obtained paclitaxel is recovered.

30. A process for preparing paclitaxel comprising reacting 2′-EE-7-Boc paclitaxel with formic acid or with a mixture of formic acid and a second organic acid in the presence or absence of a solvent wherein the solvent is immiscible in formic acid or in the mixture of formic acid and the second organic acid.

31. A process for preparing taxane intermediates of formula 1 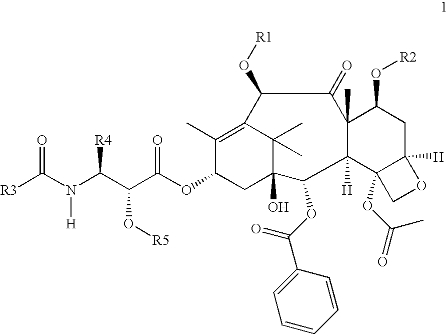
comprising reacting a 7-protected derivative of baccatin or 7,10-diprotected derivative of 10-deacetyl-baccatin (10-DAB) of formula 2 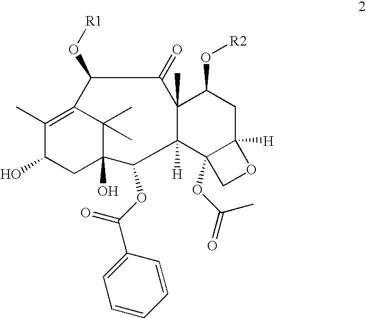
with a β-lactam of formula 3 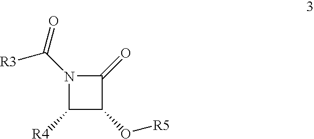
and a catalytic amount of a base, selected from the group consisting of an organometallic base, a metal hydride and mixtures thereof, providing a reaction mixture, and then quenching the reaction mixture, providing the intermediate of formula 1,
wherein
R₁, R₂ and R₅ are independently a hydroxyl protecting group;
R₃ is phenyl, substituted phenyl, a straight or branched alkyl, alkenyl, cycloalkyl, cycloalkenyl or an R₆—O— group in which R₆ is
phenyl, substituted phenyl group, a straight or branched alkyl, a straight or branched alkenyl group, a straight or branched alkynyl, a cycloalkyl, a cycloalkenyl, a bicycloalkyl substituent, or a saturated or unsaturated nitrogen; and
R₄ is a phenyl or substituted phenyl group.

32. A process for preparing a taxane comprising preparing a taxane intermediate according to the process of claim 31 and converting the obtained intermediate to the taxane.

33. The process of claim 32 wherein the taxane is paclitaxel or docetaxel.

34. The process of claim 32 comprising reacting the taxane intermediate with formic acid or with a mixture of formic acid and a second organic acid in the presence or absence of a solvent wherein the solvent is immiscible in formic acid or in the mixture of formic acid and the second organic acid.
